(19) United States
(12) Patent Application Publication
(10) Pub. No.: US 2022/0212924 A1
(43) Pub. Date: Jul. 7, 2022

(54) PRODUCTION OF SYNGAS USING RECYCLED CO2 VIA COMBINED DRY AND STEAM REFORMING OF METHANE

(71) Applicant: PYROGENESIS CANADA INC., Montreal (CA)

(72) Inventors: Ali SHAHVERDI, Verdun (CA); Pierre CARABIN, Montréal (CA)

(21) Appl. No.: 17/607,860

(22) PCT Filed: May 11, 2020
(86) PCT No.: PCT/CA2020/000056

§ 371 Date: Oct. 29, 2021

Related U.S. Application Data
(60) Provisional application No. 62/845,574, filed on May 9, 2019.

(51) Int. Cl. C01B 3/34; C07C 29/151

(52) U.S. Cl. CPC C01B 3/34; C07C 29/1518; C01B 2203/0222; C01B 2203/0883; C01B 2203/148; C01B 2203/1241; C01B 2203/061; C01B 2203/0216

(57) ABSTRACT

A process wherein CO₂, methane, and steam react at high temperatures, for instance approximately 1600° C., to form a synthetic gas or syngas. This syngas can then be used in a methanol production plant. The carbon dioxide used to produce the syngas may also comprise recovered emissions from the production of methanol or urea, such that CO₂ is recycled. The rich syngas is produced by the bi-reforming of methane, featuring a combination of dry reforming of methane and steam reforming of methane, via the reaction CO₂+3CH₄+2H₂O→4CO+8H₂, such that the H₂:CO ratio is 2. A plasma reactor may be provided for the reaction. Excess heat from the syngas may be used for heating the water that is used as steam for the reaction.

### CROSS REFERENCE TO RELATED APPLICATIONS

This application claims priority on U.S. Provisional Application No. 62/845,574, now pending, filed on May 9, 2019, which is herein incorporated by reference.

### FIELD

The present subject matter relates to the production of methanol and, more particularly, to the CO₂ resulting therefrom.

### BACKGROUND

In a conventional methanol plant, natural gas is used to produce synthesis gas or syngas, which in turn is used to produce methanol. The syngas production process is endothermic and requires large amounts of heat that is produced by burning more natural gas. This results in a process that generates large amounts of greenhouse gas emissions, mainly carbon dioxide (CO₂), from the combustion of natural gas and as a by-product from the production of syngas.

Carbon dioxide is a greenhouse gas that has a detrimental effect on human and all forms of life on the planet, causing global warming.

It would therefore be desirable to have a process that reduces the amount of carbon dioxide released to the atmosphere from the production of methanol.

### SUMMARY

It would thus be desirable to provide a process that uses carbon dioxide, for instance for the production of methanol.

The embodiments described herein provide in one aspect a process for using CO₂, comprising recuperating CO₂; and transforming the CO₂ into a synthetic gas by means of plasma.

For instance, the CO₂ used to produce the synthetic gas includes recycled CO₂ emissions from a plant.

For instance, the CO₂ emissions are obtained from a methanol producing plant.

For instance, the CO₂ emissions used to produce the synthetic gas are recycled back into the methanol production process.

For instance, the CO₂ includes emitted CO₂ from fossil-fuel heating burners used as carbon source to produce the synthetic gas.

For instance, the synthetic gas is produced via a combined plasma methane-steam reforming.

For instance, the synthetic gas is produced using a combination of dry and steam reforming of methane via the plasma, thereby producing a rich synthetic gas stream with a H₂:CO ratio of 2.

For instance, the syngas is used for the production of methanol.

For instance, CO₂ emissions from a urea plant are captured and recycled into the synthetic gas for the production of methanol.

For instance, the CO₂, methane and steam react to produce the synthetic gas, via the following reaction CO₂+3CH₄+2H₂O=4CO+8H₂.

For instance, a plasma reactor is provided for the reaction.

For instance, for the reaction, a reaction temperature of between approximately 1100-3000° C. is used.

For instance, the reaction temperature is between approximately 1100-2100° C.

For instance, the reaction temperature is between approximately 1200-1800° C.

For instance, the reaction temperature is approximately 1600° C.

For instance, part of the heat of the synthetic gas is used to heat water, which water being adapted to be at least part of the steam used to produce the synthetic gas.

For instance, a heat exchanger is provided for causing the synthetic gas to heat the water.

For instance, the synthetic gas, downstream of the synthetic gas having heated the water, is used to produce methanol.

Also, the embodiment described herein provide in another aspect a

Furthermore, the embodiment described herein provide in another aspect a process whereby CO₂ emissions from a plant are recycled by producing synthetic gas.

Furthermore, the embodiment described herein provide in another aspect a process whereby synthetic gas is produced via a combined plasma methane-steam reforming.

Furthermore, the embodiment described herein provide in another aspect a process for transformation of CO₂ to synthetic gas by means of plasma.

Furthermore, the embodiment described herein provide in another aspect a process that uses emitted CO₂ from fossil-fuel heating burners as a carbon source to produce synthetic gas.

Furthermore, the embodiment described herein provide in another aspect a process that combines dry and steam reforming of methane via thermal plasma to produce rich synthetic gas stream with a H₂:CO ratio of 2.

Furthermore, the embodiment described herein provide in another aspect a process whereby CO₂ emissions from a methanol producing plant are recycled back into the methanol production process.

Furthermore, the embodiment described herein provide in another aspect a process that combines dry and steam reforming of methane into syngas with a H₂:CO ratio of 2, required for the production of methanol.

Furthermore, the embodiment described herein provide in another aspect a process for methanol production that reduces the carbon footprint by 355 000 t CO₂ eq/yr for a 3 000 t/day methanol production plant.

Furthermore, the embodiment described herein provide in another aspect a methanol production plant integrated with a urea production plant, wherein CO₂ emissions from the urea plant are captured and recycled into syngas for the production of methanol.

Furthermore, the embodiment described herein provide in another aspect a process for producing synthetic gas using CO₂, comprising: a) providing CO₂, methane and steam; and b) submitting the CO₂, methane and steam to high temperatures so that the CO₂, methane and steam react to produce a synthetic gas.

For instance, in step b) the high temperatures are provided by plasma.

For instance, a plasma reactor is provided.

For instance, the CO₂ used to produce the synthetic gas includes recycled CO₂ emissions from a plant.

For instance, the CO₂ emissions are obtained from a production of methanol.

For instance, the synthetic gas is used in a methanol production process.

For instance, the synthetic gas is produced using a combination of dry and steam reforming of methane via the plasma, thereby producing a rich synthetic gas stream with a H₂:CO ratio of 2.

For instance, CO₂ emissions from a urea plant are captured and recycled into the synthetic gas for the production of methanol.

For instance, the CO₂, methane and steam react as per the following reaction CO₂+3CH₄+2H₂O=4CO+8H₂.

For instance, a plasma reactor is provided for the reaction.

For instance, for the reaction, a reaction temperature of between approximately 1100-3000° C. is used.

For instance, the reaction temperature is between approximately 1100-2100° C.

For instance, the reaction temperature is between approximately 1200-1800° C.

For instance, the reaction temperature is approximately 1600° C.

For instance, part of the heat of the synthetic gas is used to heat water, which water being adapted to be at least part of the steam used to produce the synthetic gas.

For instance, a heat exchanger is provided for causing the synthetic gas to heat the water.

For instance, the synthetic gas, downstream of the synthetic gas having heated the water, is used to produce methanol.

### BRIEF DESCRIPTION OF THE DRAWINGS

For a better understanding of the embodiments described herein and to show more clearly how they may be carried into effect, reference will now be made, by way of example only, to the accompanying drawings, which show at least one exemplary embodiment, and in which:

FIG. 1 is a graph showing a reaction system of CO₂+3CH₄+2H₂Oat equilibrium in accordance with an exemplary embodiment; and

FIG. 2 is a schematic block diagram of an integrated process for valorization of CO₂ from a Methanol-Urea plant in accordance with an exemplary embodiment.

### DESCRIPTION OF VARIOUS EMBODIMENTS

Generally, the process of the present subject matter is adapted to recycle CO₂ from a carbon capture system downstream of the methanol production plant into more syngas and methanol, thereby reducing the amount of CO₂ released to the atmosphere, and thus favourably reducing the greenhouse effect and global warming.

The proposed solution is based on using thermal plasma technology to valorize the CO₂ into syngas that is the main feed stream for methanol production. In this process, CO₂ is converted to syngas using a combination of dry and steam plasma reforming at high temperature. In order to be able to recycle back the CO₂ as the carbon source into the methanol or methanol-urea plant, the CO₂ should be converted to a usable product, that is syngas which consists of H₂ and CO. Dry reforming of CO₂ through reaction with methane will yield syngas with a H₂/CO ratio of 1 according to the following reaction:

CO₂+CH₄=2CO+2H₂(H₂/CO=1)

This conversion of CO₂ to syngas via the above reaction yields a syngas with a H₂/CO ratio of one (1). However, to be able to use this syngas in the methanol plant, a ratio of H₂/CO=2 is required according to the following methanol synthesis reaction:

CO+2H₂═CH₃OH

Therefore, to make it possible to reuse the excess CO₂ from the purification plant in the form of syngas, the following reaction is proposed:

CO₂+3CH₄+2H₂O=4CO+8H₂(H₂/CO=2)

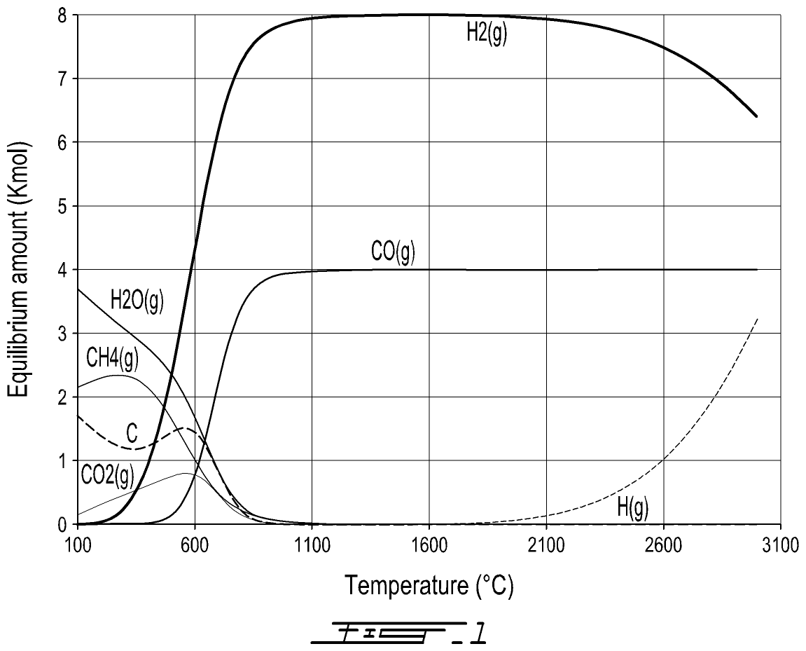

The feasibility of the above-mentioned plasma reaction was validated using HSC software that uses Gibbs-Free energy minimization method to predict the reaction system at various temperatures for a gas mixture of CO₂, CH₄, and H₂O. The ratio of CH₄ over H₂O was varied while CO₂ was kept constant until a H₂/CO ratio of 2 was produced in the reaction system, at a reaction temperature of 1600° C., which is readily archivable using plasma technology. The results of HSC calculation for a gas mixture of CO₂+3CH₄+2H₂O is shown in FIG. 1, highlighting the main products.

As can be seen in FIG. 1, the production rate of H₂ and CO maximizes at a temperature of 1600° C., and above this temperature, H₂ starts to decompose into atomic hydrogen (H) while CO is stable over a wider temperature range. For a high syngas production yield, therefore a reaction temperature of, for instance, approximately 1600° C. is recommended.

The main source of H₂O in the reaction can be steam plasma that contains a very high enthalpy and it is very reactive, which is enough for the proposed reaction to proceed at 1600° C. at a very high yield. In fact, the heat and mass (H&M) balance calculation was performed to study the specific energy required for the reaction regarding the methanol or methanol-urea plant CO₂ surplus to proceed at 1600° C. The results of H&M balance calculation are summarized in the following Table 1.

TABLE 1
Heat and Mass balance over proposed combined
plasma dry-steam reforming of CO₂ at 1600° C.

| | Temper. | Pressure | Amount | Amount | Amount | Heat Content | Total H |
|---|---|---|---|---|---|---|---|
| | ° C. | bar | kmol | kg | Nm³ | kWh | kWh |
| INPUT SPECIES | | | | | | | |
| Formula | | | | | | | |
| CH4(g) | 25 | | 3000 | 48127 | 67241 | 0.000 | −62167 |
| CO2(g) | 25 | | 1000 | 44010 | 22414 | 0.004 | −109307 |
| H2O | 25 | | 2000 | 36031 | 39 | 0.005 | −158794 |
| OUTPUT SPECIES | | | | | | | |
| Formula | | | | | | | |
| CO(g) | 1600 | | 4000 | 112040 | 89654 | 57939 | −64885 |
| H2(g) | 1600 | | 8000 | 16127 | 179309 | 108062 | 108062 |
| | | | kmol | kg | Nm³ | kWh | kWh |
| BALANCE: | | | 6000 | 0 | 179270 | 166001 | 373446 |

Assuming a theoretical 100% conversion yield of CO₂ to syngas, at a feed rate of ˜44000 kg/hr CO₂, specific energy requirement of the system is ˜373 MWhr, which gives a specific energy requirement of 2.9 kWhr/kg of syngas (H₂/CO=2). Since thermal plasma is energized by only using electricity and knowing the abundance of hydroelectric power in the Province of Quebec, Canada, the process can be considered green with near zero carbon footprint.

Since the methanol process requires syngas at a lower temperature, the excess heat that is carried by the syngas leaving the plasma reactor can be recovered. For instance, the differential energy content of the syngas stream as shown in above Table 1 with regards to a delta T of 1100° C. is ˜120 MWh, which would be enough to produce ˜160 000 kg of atmospheric pressure steam at 145° C.

In addition, there are a few important advantages of the present plasma process, as follows:
green process;
compact, as only the reactants are directly brought to the reaction temperature;
no sensitivity to the quality of CO₂ stream, as no catalyst is used; and
very high conversion yield of CO₂ to syngas, thereby resulting in a pure syngas stream.

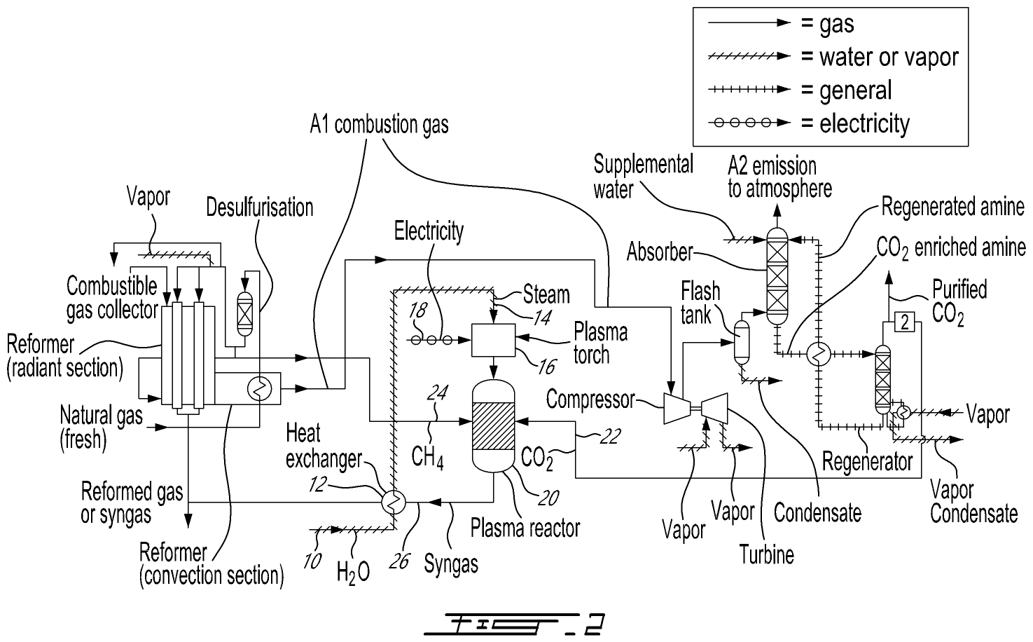

A process block diagram of the present plasma-based solution, integrated with a Methanol-Urea plant, is depicted in FIG. 2. The excess CO₂ is valorized into syngas that is then used in the methanol plant to produce methanol with 25% of its carbon by CO₂ recovery.

In summary, water is introduced at 10, which water is heater by a heat exchanger 12 and is then fed at 14 to a plasma torch 16 that is powered by electricity 18. The steam from the plasma torch 16 is fed to a plasma reactor 20, which is also fed with the aforementioned recovered CO₂ at 22 and with methane CH₄ at 24.

The syngas 26 produced by the plasma reactor 20 include excess heat, heat that the syngas does not require for its use with natural gas to produce methanol. Therefore, this excess heat in the syngas 26 is recovered in the heat exchanger 12 for heating the input water 10.

The econo-environmental impact of the present process is summarized in Table 2.

TABLE 2
Econo-Environmental impact of the present plasma-based
solution

| Specific | | | direct cost | |
|---|---|---|---|---|
| energy | GHGs | Carbon | of syngas | Physical |
| requirement | reduction | foot print | production¹ | Footprint |
| ≤2.9 kWh/kg | 355 000 t | −25% in | ^{~}0.3 $/kg | ^{~}1/10 of conven- |
| syngas | CO₂ eq/yr | methanol | | tional process, |
| | | produced | | lower CAPEX |

¹Excluding Natural gas price, and 10 cents per kWh

While the above description provides examples of the embodiments, it will be appreciated that some features and/or functions of the described embodiments are susceptible to modification without departing from the spirit and principles of operation of the described embodiments. Accordingly, what has been described above has been intended to be illustrative of the embodiments and non-limiting, and it will be understood by persons skilled in the art that other variants and modifications may be made without departing from the scope of the embodiments as defined in the claims appended hereto.

1. A process for using CO₂, comprising:
recuperating CO₂; and
transforming the CO₂ into a synthetic gas by means of plasma.

2. The process of claim 1, wherein the CO₂ used to produce the synthetic gas includes recycled CO₂ emissions from a plant.

3. The process of claim 2, wherein the CO₂ emissions are obtained from a methanol producing plant.

4. The process of claim 3, wherein the CO₂ emissions used to produce the synthetic gas are recycled back into the methanol production process.

5. The process of claim 1, wherein the CO₂ includes emitted CO₂ from fossil-fuel heating burners used as carbon source to produce the synthetic gas.

6. The process of any one of claims 1 to 5, wherein the synthetic gas is produced via a combined plasma methane-steam reforming.

7. The process of any one of claims 1 to 6, wherein the synthetic gas is produced using a combination of dry and steam reforming of methane via the plasma, thereby producing a rich synthetic gas stream with a H₂:CO ratio of 2.

8. The process of claim 7, wherein the syngas is used for the production of methanol.

9. The process of any one of claims 1 to 8, wherein CO₂ emissions from a urea plant are captured and recycled into the synthetic gas for the production of methanol.

10. The process of any one of claims 1 to 9, wherein the CO₂, methane and steam react to produce the synthetic gas, via the following reaction CO₂+3CH₄+2H₂O=4CO+8H₂.

11. The process of claim 10, wherein a plasma reactor is provided for the reaction.

12. The process of any one of claims 10 to 11, wherein, for the reaction, a reaction temperature of between approximately 1100-3000° C. is used.

13. The process of claim 12, wherein the reaction temperature is between approximately 1100-2100° C.

14. The process of claim 13, wherein the reaction temperature is between approximately 1200-1800° C.

15. The process of claim 14, wherein the reaction temperature is approximately 1600° C.

16. The process of any one of claims 1 to 9, wherein the CO₂ is transformed into the synthetic gas under temperatures of between approximately 1100-3000° C. is used.

17. The process of claim 16, wherein the temperatures are between approximately 1100-2100° C.

18. The process of claim 17, wherein the temperatures are between approximately 1200-1800° C.

19. The process of claim 18, wherein the temperatures are approximately 1600° C.

20. The process of any one of claims 10 to 15, wherein part of the heat of the synthetic gas is used to heat water, which water being adapted to be at least part of the steam used to produce the synthetic gas.

21. The process of claim 20, wherein a heat exchanger is provided for causing the synthetic gas to heat the water.

22. The process of any one of claims 20 to 21, wherein the synthetic gas, downstream of the synthetic gas having heated the water, is used to produce methanol.

23. A process whereby CO₂ emissions from a plant are recycled by producing synthetic gas.

24. A process whereby synthetic gas is produced via a combined plasma methane-steam reforming.

25. A process for transformation of CO₂ to synthetic gas by means of plasma.

26. A process that uses emitted CO₂ from fossil-fuel heating burners as a carbon source to produce synthetic gas.

27. A process that combines dry and steam reforming of methane via thermal plasma to produce rich synthetic gas stream with a H₂:CO ratio of 2.

28. A process whereby CO₂ emissions from a methanol producing plant are recycled back into the methanol production process.

29. A process that combines dry and steam reforming of methane into syngas with a H₂:CO ratio of 2, required for the production of methanol.

30. A process for methanol production that reduces the carbon footprint by 355 000 t CO₂ eq/yr for a 3 000 t/day methanol production plant.

31. A methanol production plant integrated with a urea production plant, wherein CO₂ emissions from the urea plant are captured and recycled into syngas for the production of methanol.

32. A process for producing synthetic gas using CO₂, comprising:
a) providing CO₂, methane and steam; and
b) submitting the CO₂, methane and steam to high temperatures so that the CO₂, methane and steam react to produce a synthetic gas.

33. The process of claim 32, wherein in step b) the high temperatures are provided by plasma.

34. The process of claim 33, wherein a plasma reactor is provided.

35. The process of any one of claims 32 to 34, wherein the CO₂ used to produce the synthetic gas includes recycled CO₂ emissions from a plant.

36. The process of claim 35, wherein the CO₂ emissions are obtained from a production of methanol.

37. The process of any one of claims 32 to 36, wherein the synthetic gas is used in a methanol production process.

38. The process of any one of claims 33 to 34, wherein the synthetic gas is produced using a combination of dry and steam reforming of methane via the plasma, thereby producing a rich synthetic gas stream with a H₂:CO ratio of 2.

39. The process of any one of claims 32 to 38, wherein CO₂ emissions from a urea plant are captured and recycled into the synthetic gas for the production of methanol.

40. The process of any one of claims 32 to 39, wherein the CO₂, methane and steam react as per the following reaction CO₂+3CH₄+2H₂O=4CO+8H₂.

41. The process of claim 40, wherein a plasma reactor is provided for the reaction.

42. The process of any one of claims 40 to 41, wherein, for the reaction, a reaction temperature of between approximately 1100-3000° C. is used.

43. The process of claim 42, wherein the reaction temperature is between approximately 1100-2100° C.

44. The process of claim 43, wherein the reaction temperature is between approximately 1200-1800° C.

45. The process of claim 44, wherein the reaction temperature is approximately 1600° C.

46. The process of any one of claims 40 to 45, wherein part of the heat of the synthetic gas is used to heat water, which water being adapted to be at least part of the steam used to produce the synthetic gas.

47. The process of claim 46, wherein a heat exchanger is provided for causing the synthetic gas to heat the water.

48. The process of any one of claims 46 to 47, wherein the synthetic gas, downstream of the synthetic gas having heated the water, is used to produce methanol.
